(19) United States
(12) Patent Application Publication
(10) Pub. No.: US 2011/0264055 A1
(43) Pub. Date: Oct. 27, 2011

(54) Medical Device

(76) Inventors: Andreas Grundlehner, Zumikon (CH); Melissa Matarese, Franklin Lakes, NJ (US)

(21) Appl. No.: 12/988,997

(22) PCT Filed: Apr. 23, 2009
(86) PCT No.: PCT/EP09/54863

§ 371 Date: Jan. 4, 2011

(30) Foreign Application Priority Data
Apr. 23, 2008 (US) 61047117

(51) Int. Cl. A61M 31/00

(52) U.S. Cl. 604/264

(57) ABSTRACT

The present invention is related to a medical device for transvaginal delivery, comprising a flexible coil which is able to expand from a compressed state after being released so that it comes into contact with the vaginal mucosa, said coil carrying a medicament. The present invention is furthermore related to a system comprising a tampon which is wound with said medical device.

The present invention is related to a device, in particular a coil optionally wound around a tampon, for vaginal application.

Tampons have been in use for decades in the field of female hygiene. While their predominant use is the absorption of blood or other body liquids during the periods of menstruation, there have been some reports about therapeutic applications of tampons.

Many women experience mild discomfort during menstruation, but some women suffer severe pain during their periods. Such painful menstruation is called Dysmenorrhoea. Dysmenorrhoea can be so pronounced that the affected women are prevented from carrying out their normal activities and have to take medication. It is assumed that these painful episodes are evoked by changes in hormonal levels during the menstrual cycle. Moreover, other physiological conditions such as the premenstrual syndrome or menopause are sometimes associated with painful events.

It has been found that these painful events can be alleviated by administration of drugs which inhibit the prostaglandin synthesis in the body. These drugs are well-known. The most prominent class of suitable drugs are the non-steroidal anti-inflammatory drugs (NSAIDs), for example ibuprofen, naproxen, mefenamic acid, celecoxib acid, acetyl salicylic acid (Aspirin®) or rofecoxib.

It is of course possible to take such medicaments orally, as they are available as tablets. However, since during menstruation women already have to use a sanitary device such as a napkin or a tampon, it would be more convenient if both the sanitary device as well as the medicament could be applied simultaneously in one application step. Thereby, also any risk of forgetting to take the medicament would be avoided.

Administering the medicament via the transvaginal route has several advantages: The drug is directly applied at the location where it is needed (local treatment), which results in a higher concentration of the drug at said site, as well as results in a quicker onset of the medicament's effect. Since the drug is taken up into the blood circulation system, the transvaginal route may provide both a local and systemic effect of the administered drug. Moreover, by said administration route the so-called first pass effect is avoided: A drug which is taken orally undergoes a first-pass metabolism once it has reached the small intestine. This means that at least a portion of the drug is transferred to the liver and optionally metabolized, before it is released once again. The first-pass effect results in a smaller onset of the medicament's action as well as in a decreased efficiency of the drug (due to metabolism). Also, food effects have to be taken into consideration for orally administered drugs.

The transvaginal administration avoids those drawbacks and complications. It is an easily accessible and convenient administration route. It is non-invasive, i.e. the drug has not to be administered by e.g. using a syringe. Moreover, the vaginal epithelial tissue is particularly suited for the uptake of medicaments, since it exhibits an extensive blood supply and thus guarantees a rapid uptake of an administered drug. The vaginal mucosa exhibits a significant permeability for many common drugs, especially for NSAIDs.

Several devices, such as pessaries or tampons, have been used for local transvaginal delivery of medicaments. A major application is the delivery of contraceptive drugs, but the administration of other drugs such as NSAIDs has also already been suggested.

For example, in WO 03/063829 A1 a medicated tampon is suggested which comprises a dosage form. Most preferably, said dosage form is provided at the tip of the tampon. However, said tampon is somewhat difficult to manufacture. Moreover, the drug provided at the tip of the tampon does not directly contact the vaginal mucosa and is thus not optimally taken up. Furthermore, drug and medicament are connected with each other and have to be taken together. For applications where a tampon is not needed, such as in the treatment of menopausal conditions, said device is thus not the best choice.

In U.S. Pat. No. 7,004,171 B2, a system for transvaginal drug delivery is suggested which consists of a tampon having deposed on a part of its surface a drug delivery system. Also here, the tampon and the medicament are tightly connected with each other, which results in non-optimal kinetic characteristics of the drug uptake. Moreover, the portion of the tampon's surface which contains the drug delivery system is not permeable for body liquids which should be absorbed by the tampon. Therefore, the tampon's actual purpose is deteriorated.

In EP-0 594 628 B1, a tampon or a sanitary napkin is suggested which is impregnated with a culture of living lactic acid producing bacteria. However, said impregnation also somewhat compromises the function of the tampon or napkin of absorbing blood or other body liquids.

It was therefore the object of the present invention to provide a medical device for transvaginal drug delivery, which provides a greater degree of freedom with respect to its application, i.e. with or without a sanitary device, and which provides an improved uptake of the medicament through the vaginal mucosa.

This object was solved according to the present invention by a medical device for transvaginal delivery, comprising a flexible coil which is able to expand from a compressed state after being released so that it comes into contact with the vaginal mucosa, said coil carrying at least one medicament.

The coil of the present invention provides a large surface area with defined contact with the vaginal mucosa and therefore provides a predictable drug delivery profile, in contrast to prior art solutions where the drug is released into the vagina and its bioavailability suffers from diffusion inconsistencies.

According to the present invention, the term “coil” designates a sleeve-like, spirally wound device having such a diameter and length that it suitably fits into a woman's vagina. The pitch height of the coil may vary. According to the present invention, the pitch height is preferably chosen such that the entire coil comprises 1 to 30, preferably 2 to 20 and most preferably 3 to 10 windings. Preferably, each coil winding has a thickness of between 0.1 and 1.0 cm, more preferably between 0.1 and 0.5 cm.

According to the present invention, it is preferred that the coil has a band-like cross-section, thus increasing the surface of the coil which may come into contact with the vaginal mucosa.

The coil of the present invention is flexible. In other words, the coil can be compressed and stored in a container in a compressed state. Upon release from said container, the coil radially expands from said compressed state into a state of relaxation such that the coil comes into direct contact with the vaginal mucosa. This brings about a significant advantage of the device of the present invention: If the coil is inserted, for example, into a woman's vagina while being compressed within a container, upon being released from said container the coil will expand and come into direct contact with the vaginal mucosa. Thus, the medicament carried by said coil comes also into close vicinity or contact with the vaginal mucosa and is taken up more completely and more quickly as compared to devices from the prior art where no such contact between medicament and mucosa is established. According to one embodiment of the present invention, said coil has a spring-like characteristic which automatically enables said coil to radially expand from a pre-tensioned state.

The skilled man is aware of how to make flexible coils. Preferably, the coil is made of a material which itself is flexible. Such materials which can be used in the present invention are known to the skilled man. Preferably, a flexible polymeric material may be used.

According to a preferred embodiment of the present invention, the coil is made from a hydrogel. Hydrogels are hydrophilic three-dimensional matrices which can be used as carrier materials. Hydrogels are polymers which swell in an aqueous environment. Due their high water content, hydrogels resemble natural living tissue. They have a three-dimensional structure, wherein the molecules are connected with each other by e.g. ionic, hydrogen or covalent bonds. Within said structure, active agents such as drugs can be immobilized and released under controlled conditions. The hydrogel according to the present invention has to be sufficiently mechanically and chemically stable under conditions which occur in the female vagina. The mechanical and chemical strength of a hydrogel can be adjusted by e.g. incorporating crosslinking agents or co-monomers into the hydrogel. On the other hand, a too great degree of crosslinking in the hydrogel should be avoided, since then the elasticity and thus the flexibility of the material would be deteriorated.

Of course, the hydrogels to be used in the present invention must be biocompatible, i.e. non-toxic. Generally, hydrogels can be neutral or ionic, homopolymers or copolymers. The methods of polymerization are well-known to the skilled man and need not be reiterated here. The hydrogels of the present invention may be formed from monomers which are commonly used for the manufacture of hydrogels. Examples of suitable monomers include HEMA (hydroxyethyl methacrylate), HEEMA (hydroxyethoxyethyl methacrylate), HDEEMA (hydroxydiethoxyethyl methacrylate), MEMA (methoxyethyl methacrylate), MEEMA (methoxyethoxyethyl methacrylate), MDEEMA (methoxydiethoxyethyl methacrylate), EGDMA (ethylene glycol dimethacrylate), NVP (N-vinyl-2-pyrrolidone), VAC (vinyl acetate), AA (acrylic acid), HPMA (N-2-hdyroxypropyl methacrylamide), EG (ethylene glycol), PEG (polyethylene glycol), PEGA (polyethylene glycol acrylate), PEGMA (polyethylene glycol methacrylate), PEGDA (polyethylene glycol diacrylate), and PEGDMA (polyethylene glycol dimethacrylate).

According to the present invention, also other polymeric materials such as, for example, vinyl acetate polymers, polysaccharides (e.g. cellulose, hyaluronic acid and dextran), polyester amides, polyols and poly acids, (e.g. using monomers such as sebacic acid, citric acid, lactic acid, glycolic acid, xylitol, maltitol, glycerol, amino alcohols, glutamic acid, aspartic acid etc.) may be used.

Hydrogels may be formed, when necessary, by functionalisation of the polymer with organic groups (e.g. acrylate, aldehyde, amine, carboxylic groups) to promote the desired degree of crosslinking for gel formation via chemical methods, ultraviolet light methods or heat methods. Thus, the elastic mechanical properties necessary to expand the coil, adhesiveness of the coil to the mucosa and the desired controlled drug retention during storage and release when placed in vivo may be achieved. Multiple polymer layering of different polymers may allow for greater control over each of these goals.

According to another embodiment of the present invention, the coil may also have a multi-layer structure, wherein the multiple layers may be the same or different. For example, the coil may have a solid core layer which is surrounded or covered by one or more layers of a hydrogel as described above. The solid core may be made of any material described above, for example of a flexible polymeric material. The only requirement is that the resulting coil has to possess sufficient flexibility such that it expands upon release in the vagina to such an extent that it comes into direct contact with the vaginal mucosa.

The coil of the invention carries a medicament. According to the present invention, “carries” means in this respect that the medicament is connected with the coil so that it remains at its location during storage and transport. For example, the medicament may be deposited on or attached to the surface of the coil. For example, the medicament may be covalently bonded to functional groups which are present on the surface of the coil. Preferably, the resulting bonds are of such nature that they degrade under physiological conditions (e.g. change of pH level, enzymatic cleavage). In the case of a hydrogel, the medicament may be immobilized or entrapped within said hydrogel and controllably released during swelling of the gel therefrom. Conditions of drug entrapment into or onto the polymer or hydrogel structures are mild and do not cause a change in the drug structure or function.

The coil should preferably be designed such that it retains mechanical strength and structure for the duration of time it is left in the body (which will depend on the application e.g. for alleviation of dysmenorrhoea, 6-8 hours) such that it can be easily removed after the specified application time. The polymer structure and any released monomers or chemical moieties from crosslinking bonds with drugs or within the polymer are biocompatible and have no adverse effects.

In principle, the coil of the present invention may carry any medicament. It is of course preferred to use medicaments which should exhibit a local effect in the vaginal region of the woman's body. For example, for the treatment of pains occurring during the menstrual periods, an anti-inflammatory agent such as a NSAID is used. A preferred medicament to be used according to the present invention is Ibuprofen. Alternatively, also local anesthetic drugs such as Lidocaine may be used.

According to the present invention, the following non-limiting examples of bioactive compounds may be used:

NSAIDs such as Aspirin, Ibuprofen, Indomethacin, Phenylbutazone, Bromfenac, Fenamate, Sulindac, Nabumetone, Ketorolac, Diclofenac and Naproxen;

Local anesthetics such as Lidocaine, Mepivacaine, Etidocaine, Bupivacaine, 2-Chloroprocaine hydrochloride, Procaine, and Tetracaine hydrochloride;

Calcium channel antagonists such as Diltiazem, Israpidien, Nimodipine, Felodipine, Verapamil, Nifedipine, Nicardipine, and Bepridil;

Potassium channel blockers such as Defetilide, E-4031, Almokalant, Sematilide, Ambasilide, Azimilide, Tedisamil, RP58866, sotalol, Piroxicam, and Ibutilide;

β-adrenergic agonists such as Terbutaline, Salbutamol, Metaproterenol, and Ritodrine;

Vasodilators such as nitroglycerin, isosorbide dinitrate and isosorbide mononitrate, or PDE inhibitors or guanylatecyclase activators;

COX-2 inhibitors such as Celecoxib, Meloxicam and Flosulide;

Hormone replacement drugs such as estrogen, estradiol and progestogen; Androgenic hormones such as Testosterone.

These drugs may be used either alone or in combination.

The amount of drug which is to be carried by the coil depends on the indication which is to be treated. The skilled man can readily determine the required amount of the drug for a specific indication.

The skilled man is aware of how to deposit or attach active agents onto a surface of a material such as a polymer material. In particular, the skilled man is aware of how to prepare a hydrogel which is loaded with an active agent.

According to one embodiment of the present invention, the coil may comprise several portions which are divided from each other. The separate portions can be, for example, loaded with different drugs in order to even improve the therapeutic effect of the device. For example, one portion of the coil may be loaded with one NSAID as described above, whereas another portion of the coil may carry a local anesthetic drug, such as lidocaine.

Alternatively, the concentration of the at least one medicament attached to the coil may vary along the length of the coil. For example, at the upper end of the coil a higher concentration of drug may be present than at its lower end.

The coil of the present invention furthermore comprises a string which is preferably attached to the lower end (i.e. the end of the coil which is directed to the outer opening of the vagina) of the coil. It is also possible to attach said string to the other, upper end of the coil, and guide the string through the coil. By means of said string the coil can be pulled out of the vagina after use. The string can be made of any material which is suitably used for strings which are attached to tampons for pulling them out of the vagina. The string has to be affixed to the coil in such a way that it does not tear apart when a pulling force is exerted onto said string. For example, the string may tightened to the coil by means of a knot. According to one embodiment, the coil may possess a rear end which is designed to be tightly connected with said string.

The coil of the present invention is preferably stored within a container in a compressed state. Said container is most preferably construed like an applicator for a tampon. Such applicators are well-known and consist of a tube-shaped protector member and an applicator member.

The tube-shaped protector member can be in the form of a spirally wound, convolutedly wound or longitudinally seamed hollow tube which is formed from paper, paperboard, cardboard, or other suitable material, or a combination thereof. The protector member can also be in the form of a seamless polymeric tube. Said polymer is preferably polyethylene, but may be also polypropylene or other suitable polymer. The protector member, also commonly referred to as an outer tube, can be of any suitable dimensional arrangement. For example, the protector member may be fairly rigid and have a relatively small diameter of about 10 mm to about 20 mm. The walls of the protector member can be smooth and/or slippery to facilitate expulsion of the coil and to facilitate insertion of the protector member into a woman's vagina, respectively. The protector member can be coated to give it a high slip characteristic. Wax, polyethylene, a combination of wax and polyethylene, cellophane and clay are representative coatings that can be applied to the protector member to facilitate comfortable insertion. The inside diameter of the protector member is usually less than about 20 mm and preferably less than about 15 mm. Although the exterior diameters of tampons vary, most tampons utilized by women have an external diameter of less than about 20 mm.

The protector member can be a straight, elongated cylindrical tube. It is also possible to form the protector member into an arcuate shape. The arcuate or curved shape can assist in providing comfort when inserting the protector member into a woman's vagina.

The applicator member or inner tube can be a spirally wound, a convolutedly wound or a longitudinally seamed hollow tube constructed from paper, paperboard, cardboard, or other suitable material, or a combination thereof. The applicator member can also be in the form of a seamless polymer tube, as the protector member. The applicator member can be constructed of the same material as the protector member, or it can be made out of a different material. The applicator member may also be a solid stick or use some other unique shape. The applicator member has a bit a smaller diameter, so that it can be moved into the protector member.

The applicator member can be pushed into the protector member, thereby releasing the coil from the protector member. For the smooth release of the coil, the upper end of the protector member (which is first inserted into the vagina) comprises a plurality of pleats or petals that can radially open such that in the open state this end of the protector member has around the same diameter as the remainder of the protector member.

When the applicator member is pushed into the protector member, the coil pushes from the interior against said pleats or petals and forces them to open. After the applicator member has been sufficiently pushed into the protector member, the coil is released from the protector member into the vagina. Since the coil is stored in the protector member in a compressed state, upon release into the vagina the coil will expand. In case of the preferred embodiment of the present invention where said coil comprises a hydrogel material, said expansion will be enhanced by the swelling of the hydrogel in the aqueous environment within the vagina. Due to said expansion, the coil comes into direct contact with the vaginal mucosa and thus ensures an improved transvaginal drug delivery.

In order to alleviate the pushing of the applicator member into the protector member, those members may exhibit fingergrips.

According to the present invention, also a system is provided wherein the coil is wound around a tampon. This is particularly preferred for the treatment of conditions which occur in connection with the menstruation. The tampon absorbs the body fluids, while the coil releases the drug for the alleviation of e.g. Dysmenorrhoea.

Tampons have been known for more than 70 years and need not be described in detail here. According to the present invention, any common tampon may be used. Such tampons include an absorbent material. The absorbent material can be formed from fibres that are assembled into an absorbent sheet or ribbon. Alternatively, the absorbent material can be formed from absorbent fibres that are assembled and compressed into a generally elongated and/or cylindrical configuration. The absorbent material is desirably formed from natural cellulosic fibre, such as cotton and rayon. For example, the absorbent material can be 100% cotton, 100% rayon, a blend of cotton and rayon fibres, or other materials known to be suitable for tampons, including artificial fibres such as polyester, polypropylene, nylon or blends thereof. The absorbent material may also include degradable fibres.

Tampons suitable for use in this invention are usually made of absorbent fibres, including one or both of natural and synthetic fibres, compressed into a unitary body of a size that may easily be inserted into the vaginal cavity. Tampons are normally made in an elongated cylindrical form in order that they may have a sufficiently large body of material to provide the required absorbing capacity, but may be made in a variety of shapes. The tampon is typically compressed. Compression may be achieved by predominantly longitudinally-, axially-, or radially-applied pressure, or a combination thereof.

The coil is wound around the tampon such that the body fluids have enough accessability to the tampon itself, so that they can be readily absorbed. In other words, the coil is wound around the tampon with such a pitch height, for example that the coil is wound 3 to 5 times around the tampon, that there is sufficient space between the individual coil windings where body fluids can access the underlying tampon. This is an advantage of the system of the present invention.

According to one embodiment of the present invention, a gap may be provided between the coil and the tampon, thus ensuring an even better access of body fluids to the tampons.

Moreover, another advantage of the system of the present invention resides in the fact that the structure of the tampon is supported by the coil wound around the tampon. Therewith, the likelihood is lessened that any fibre from the tampon may be torn off and remain in the vaginal cavity, where it can cause irritations.

According to this embodiment of the present invention, the above described string for pulling the device out of the vagina may be either attached to the lower end (i.e. the end of the coil which is directed to the outer opening of the vagina) or to the other, upper end of the coil, as described above. Alternatively, the string may be attached to the tampon. The string may be attached to the lower end (i.e. the end of the tampon which is directed to the outer opening of the vagina) of the tampon. Alternatively, the string may be attached to the other, upper end of the tampon and guided through the entire tampon so that it exits the tampon at its above described lower end.

Also here, the string can be made of any material which is suitably used in the art for such strings. The string has to be affixed to the tampon in such a way that it does not tear apart when a pulling force is exerted onto said string.

The coil may be wound around the tampon by any respective commonly used method. For example, a string of hydrogel material may be formed by a suitable method, e.g. by an extrusion method, and subsequently said string is wound around the tampon.

The resulting system of tampon and coil is subsequently compressed and transferred into the above described container, where it is stored in a compressed state. Upon release from the container into the vagina, the swelling of the hydrogel and the expansion of the compressed tampon material supports the expansion of the system such that the coil comes into direct contact with the vaginal mucosa, thus ensuring regular and consistent delivery of the drug through the vaginal mucosa.

While according to the present invention it is most preferred to use a coil as a medical device, also other devices which fulfil the same function may be used. The medical device may comprise an expandable member having a radially flexible elastic tubular body. For example, this tubular or sleeve-like body can be composed of a plurality of flexible and elastic thread elements which define a radially self-expanding body. The expanded state of the body mainly depends on the inherent rigidity of the threaded elements. Another expandable member conceivable to solve the object of the present invention would be a U-shaped clamp which is preferably in a pre-stressed state. This clamp may consist of two limbs extending in axial direction along the cylindrical surface area of a tampon. Alternatively, the expandable member may comprise a radially flexible tubular body in a mesh-like configuration which may radially expand in a passive way forced by the expanding tampon after insertion into the vagina. It can be seen from this embodiment that it is not compulsory for certain purposes that the expandable member must be in a compressed state before being released.

The device of the present invention is suitable for alleviating painful conditions which occur in the vaginal region. For the treatment of conditions which are not associated with the menstrual period, the coil may be used alone without a tampon. For the treatment of conditions which are associated with the menstrual period, the coil is preferably used as a system together with a tampon, so that both the absorption of body fluids as well as the alleviation of pain can be carried out with one device.

If the device of the present invention is to be used for the treatment of yeast infections (caused by candida), antimycotic active ingredients such as clotrimazol, Amphotericin B, Tetracyclintrihydrat, Nystatin, Providon-Jod, Metronidazol, Candicidin or Natamycin (Pimaricin) are applied locally by means of the device of the invention. If there is an additional need for the treatment of bacterial infections, active ingredients such as Tetracyclinhydrochlorid, Dequaliniumchlorid, or Prednison are applied locally by means of the device of the invention, optionally in combination with lactic sugar which promotes the renewal of the natural milieu in the vagina.

If the device of the present invention is to be used for the treatment of bacterial infections, active ingredients such as Clindamycinhydrochlorid, Metranidazol, Tinidazol, ascorbic acid (vitamin C), lactic acid and glycogen, or clindamycin phosphat are applied locally by means of the device of the invention.

Principally, the device of the present invention could also be used for the treatment of viral infections, for the treatment/alleviation of the symptoms of menopause (such as dry mucosa), post-surgical treatment as well as for the prophylaxis and treatment of cancers, in particular cervical cancer.

Usually, the device and/or system of the present invention is inserted into the vagina and maintained there for about 4 to 6 hours.

The present invention will be further explained with reference to non-limiting drawings.

FIG. 1 shows a first embodiment of the system of the present invention comprising a coil which is wound around a tampon.

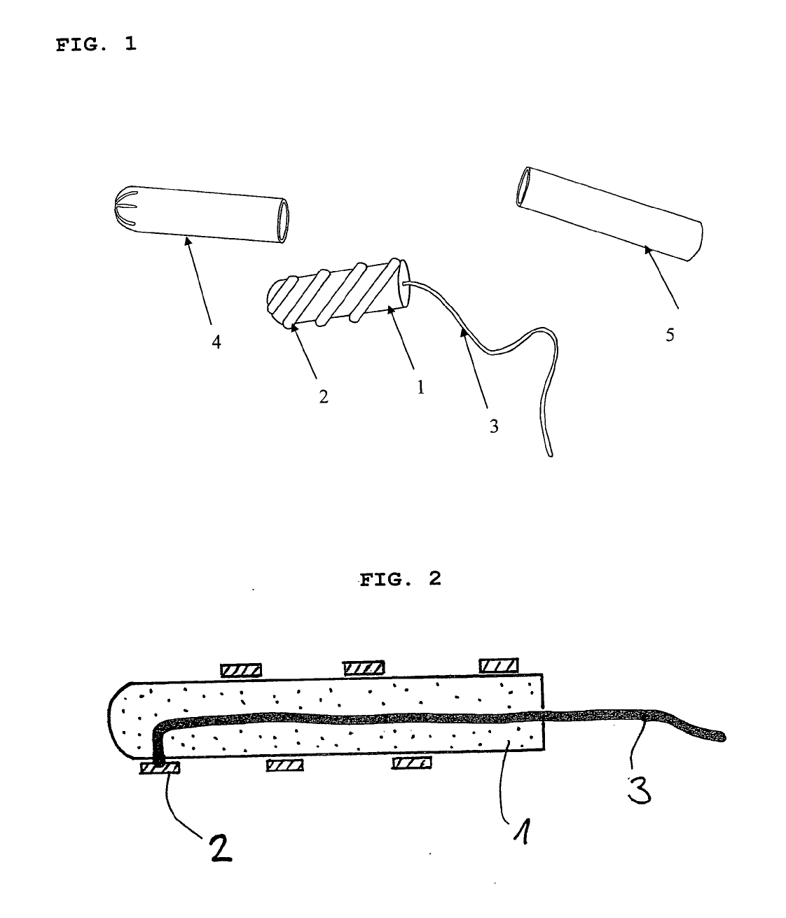

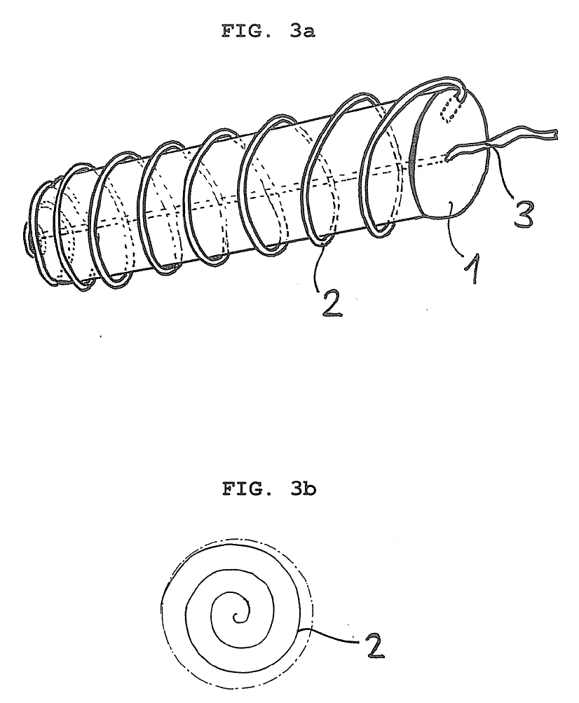

FIG. 2 shows a cross-sectional view of the embodiment of FIG. 1.

FIG. 3a shows another embodiment of the system shown in FIG. 1, wherein the coil comprises a specific coil having at least one closed end.

FIG. 3b shows a cross-sectional view of the coil of FIG. 3.

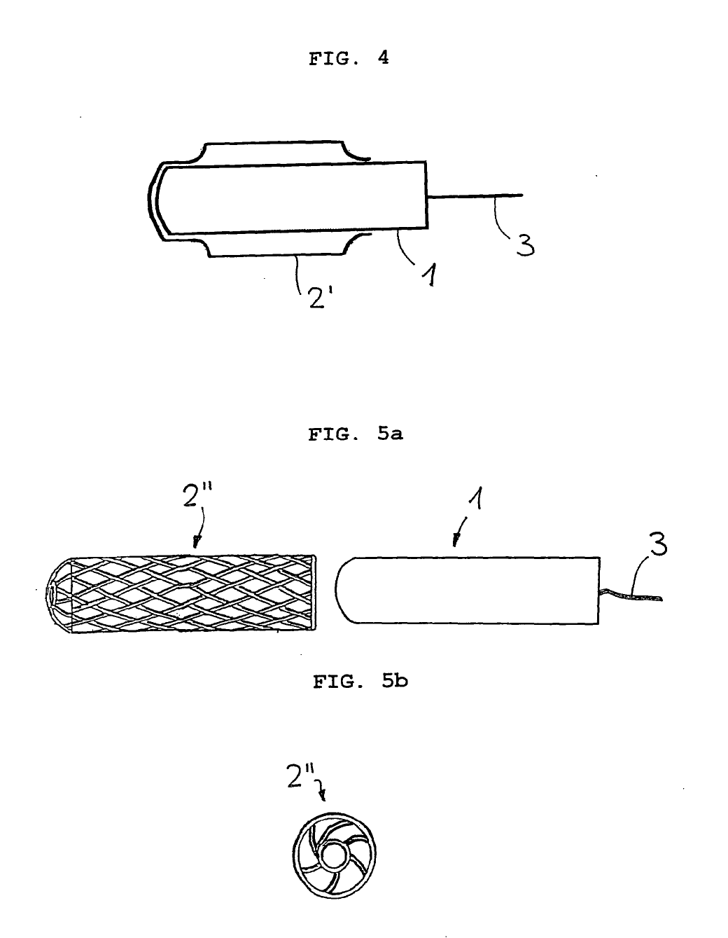

FIG. 4 shows another embodiment of the system of the present invention, comprising a expandable member clamped onto a tampon.

FIG. 5a shows another embodiment of the system of the present invention, comprising a radially flexible tubular member in a mesh-like configuration surrounding a tampon.

FIG. 5b shows a front view of the tubular member of FIG. 5a.

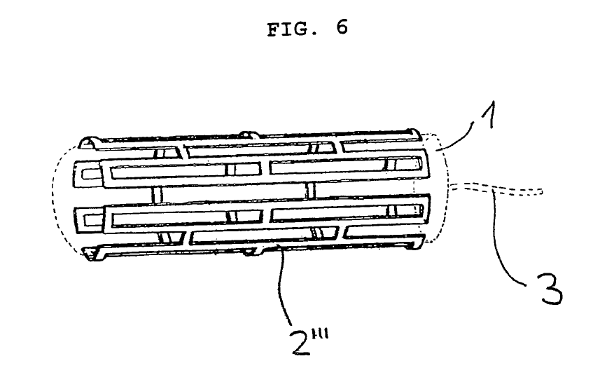

FIG. 6 shows another embodiment of the system of the present invention, comprising a radially flexible tubular member composed of a plurality of thread elements surrounding a tampon.

In FIG. 1 an embodiment of the system of the present invention is shown. A conventional tampon 1 in the form of a cylindrical plug is provided. Said tampon 1 is wound with a coil 2. The coil 2 is made of a hydrogel from PHEMA. The coil 2 has a thickness of about 0.3 cm and is wound 4 times around the tampon 1. A string 3 is attached to the lower end of the coil 2. Within the hydrogel coil 2, an effective amount of ibuprofen is immobilized and can be controllably release through the vaginal mucosa upon direct contact of the coil 2 with the mucosa.

Also shown in FIG. 1 is a container consisting of a protector member 4 and an applicator member 4. The system of tampon 1 and coil 2 is stored in a compressed state within said protector member 4 and can be pushed out of said protector member 4 by moving the applicator member 5 into said protector member 4.

The schematic FIG. 2 shows a cross section of a system comprising a tampon 1 which is wound with a coil 2. As can be seen, the segments of the coil 2 have a rectangular cross sectional area. Such coils made from a band material can improve the medicinal effect of the device of the present invention. However, other forms of the coil are also conceivable, such as coils having circular cross sectional areas. The string 3 is fixed to a distal end of the coil 2 and runs from the distal end to a proximal end inside the coil 2.

A further embodiment of the system of the present invention is shown in FIG. 3a. At the distal end of the tampon 1, the coil 2 is closed. This closure is defined by a tapered portion adjoining a sleeve-like or tubular portion of the coil 2. FIG. 3b shows a front view of said tapered portion of the coil 2. On the opposite side of the tampon 1, the coil 2 is fixed by a hook-shaped free end of the coil 2 penetrating the tampon. However, it is also conceivable that the coil is closed on both sides in a manner as shown in FIG. 3b. In this case the coil would define a cage in which the tampon is securely held within the coil. The string 3 is fixed to the distal end at the tapered portion of the coil 2.

FIG. 4 shows an expandable member 2′ docked on a tampon 1 in exploded view. The member 2′ is designed as a U-shaped clamp which is in a pre-stressed state. As can be seen, the clamp 2′ consists of two limbs extending in axial direction along the cylindrical surface area of a tampon 1.

In FIG. 5a, the expandable member 2″ has a radially flexible tubular body in a mesh-like configuration. The tubular body is able to radially expand in a passive way forced by the expanding tampon after insertion into the vagina. In FIG. 5b, a front view of the distal end of the expandable member 2″ can be seen.

The embodiment of the system as shown FIG. 6 comprises an expandable member 2′″ with a radially flexible elastic tubular body composed of a plurality of flexible and elastic thread elements which define a radially self-expanding body.

The invention has been described with reference to various specific and illustrative embodiments and techniques. However, it should be understood that many variations and modifications may be made while remaining within the spirit and scope of the invention.

Accordingly, this invention is intended to embrace all such alternatives, modifications and variations that fall within the spirit and scope of the appended claims.

1. Medical device for transvaginal delivery, comprising a flexible coil which is able to expand from a compressed state after being released so that it comes into contact with the vaginal mucosa, said coil carrying at least one medicament.

2. Medical device according to claim 1, wherein said coil is made from a hydrogel.

3. Medical device according to claim 2, wherein said hydrogel is made from monomers selected from the group consisting of hydroxyethyl methacrylate, hydroxyethoxyethyl methacrylate, hydroxydiethoxyethyl methacrylate, methoxyethyl methacrylate, methoxyethoxyethyl methacrylate, methoxydiethoxyethyl methacrylate, ethylene glycol dimethacrylate, N-vinyl-2-pyrrolidone, vinyl acetate, acrylic acid, N-2-hydroxypropyl methacrylamide, ethylene glycol, polyethylene glycol, polyethylene glycol acrylate, polyethylene glycol methacrylate, polyethylene glycol diacrylate, and polyethylene glycol dimethacrylate.

4. Medical device according to claim 1, wherein said coil has a multi-layer structure.

5. Medical device according to claim 4, wherein said multi-layer structure comprises a solid core which is surrounded by at least one layer of a hydrogel.

6. Medical device according to claim 1, wherein said coil carries an anti-inflammatory agent, or a local anesthetic drug, or a combination thereof.

7. Medical device according to claim 6, wherein said anti-inflammatory agent is a non-steroidal anti-inflammatory drug.

8. Medical device according to claim 1, further comprising a string which is attached to said coil.

9. Medical device according to claim 1, further comprising a container in which said coil is stored in a compressed or pre-tensioned state.

10. Medical device according to claim 9, wherein said container comprises a tube-like protector member and an applicator member which can be pushed into said protector member.

11. System, comprising a tampon which is wound with a medical device according to claim 1.

12. System according to claim 11, wherein said tampon is stored in a compressed state in a container.

13. System according to claim 11, wherein said container comprises a tube-like protector member and an applicator member which can be pushed into said protector member.

14. System according to claim 11, further comprising a string which is attached to said coil or to said tampon.
